(19) United States
(12) Patent Application Publication
(10) Pub. No.: US 2006/0178749 A1
(43) Pub. Date: Aug. 10, 2006

(54) Modular porous implant

(75) Inventors: John E. Pendleton, Fort Wayne, IN (US); John E. Meyers, Columbia City, IN (US); Rohit K. Somani, Winona Lake, IN (US)

Correspondence Address: ZIMMER TECHNOLOGY - REEVES, P. O. BOX 1268, ALEDO, TX, 76008, US

(73) Assignee: Zimmer Technology, Inc.

(21) Appl. No.: 11/055,322

(22) Filed: Feb. 10, 2005

(51) Int. Cl. A61F 2/38; A61F 2/28; A61F 2/30

(52) U.S. Cl. 623/20.15; 623/20.28; 623/23.39; 623/23.5

(57) ABSTRACT

A modular porous implant and method of intraoperative assembly are presented.

### FIELD OF THE INVENTION

The present invention relates to surgical implants. In particular, the present invention relates to modular porous implants for surgical repair of skeletal joints.

### BACKGROUND

Degenerative and traumatic damage to the articular cartilage and other structures of skeletal joints can result in pain and loss of function of the joint. Both prosthetic joint replacement with an articulating implant as well as joint fusion with a joint immobilizing implant have been used to relieve pain and/or restore joint function. For example, joint replacement surgery is frequently utilized to alleviate pain and restore joint function by cutting away the damaged portions of the joint and replacing them with prosthetic components. For example, in knee joint replacement surgery, a femoral implant is mated with the cut end of the femur and a tibial component is mated with the cut end of the tibia such that the two components articulate with one another.

The use of modular surgical components has become popular because it allows the surgeon to assemble components in a variety of configurations at the time of surgery to meet specific patient needs relative to size and geometry. For example, modular joint replacement components may be provided having separate anchorage components and articulating components to allow the surgeon flexibility in assembling a variety of configurations of bone coverage, component thickness, kinematic constraint, and bone attachment mechanism. For example, a modular tibial component may include separate tray and bearing components in a variety of sizes and shapes that can be combined intraoperatively. Modular surgical components typically include rigid metal bases and separate modular components mechanically joined to the base with dovetails, bolts, or clips.

### SUMMARY

The present invention provides a modular porous implant.

In one aspect of the invention, first and second implant components each include a porous surface. An intermediate material is intraoperatively positionable between the porous surfaces in a fluid state to interdigitate with the porous surfaces to join them.

In another aspect of the invention, a knee joint implant system includes a femoral implant and a tibial implant. The tibial implant includes a tibial tray component and an articular surface component. The tibial tray component includes a porous upper surface and the articular surface component includes a porous lower surface. An intermediate material is intraoperatively positionable between the porous surfaces in a fluid state to interdigitate with the porous surfaces to join them.

In another aspect of the invention, method for assembling a modular porous implant includes: intraoperatively positioning an intermediate material between first and second implant components; interdigitating the intermediate material in a fluid state with a porous surface on each of the first and second implant components; and intraoperatively transitioning the intermediate material from a fluid state to a solid state to join the first and second implant components.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is an exploded perspective view of an illustrative example in the form of a knee implant system including a femoral implant and a modular tibial implant having an articular surface component and a tibial tray component according to the present invention;

FIG. 2 is a bottom plan view of the articular surface component of FIG. 1;

FIG. 3 is a top plan view of the tibial tray component of FIG. 1;

FIG. 4 is a cross sectional view of the articular surface component and the tibial tray component of FIG. 1 assembled together; and

FIG. 5 is a perspective view of a knee joint depicting the implantation of the implants of FIG. 1.

### DESCRIPTION OF THE ILLUSTRATIVE EXAMPLES

Examples of the present invention include modular porous implants for surgical repair of skeletal joints. The implants may be used to repair joints of the hip, knee, shoulder, spine, elbow, wrist, ankle, digits, and/or other skeletal joints. The implants may include both articulating implants for prosthetic joint replacement as well as joint immobilizing implants for joint fusion. The implants may include separate anchorage components and/or articulation components that may be joined together at the time of surgery. The components may be joined outside of the patient's body and subsequently implanted as an assembly. Alternatively the components may be individually implanted and subsequently joined. The components may be sized and shaped to facilitate a minimally invasive surgical technique in which the individual components are inserted through a small incision and subsequently assembled. For example, the components may include a tibial tray component for anchoring to a tibial bone and an articular surface component for supporting knee joint articulation with a femur. The tibial tray component and articular surface component may be joinable intraoperatively and within the surgical wound.

The modular components may be joined by providing opposing porous surfaces on each of the components and interposing an intermediate material between the surfaces. The porous surfaces may include macro and/or micro porous surfaces including beads, non-woven fibrous structures, woven structures, plasma sprayed structures, machined undercuts, vapor deposited structures, and/or other suitable porous surfaces and combinations thereof. The porous surfaces may include metals, polymers, ceramics, and/or other suitable materials and combinations thereof. For example, the porous surfaces may include tantalum vapor deposited in a porous configuration resembling trabecular bone.

The intermediate material may include metals, ceramics, polymers, and/or other suitable materials and combinations thereof. The intermediate material may be transformable between a fluid state and a solid state such that it may be interdigitated with pores in the modular components in a fluid state and then solidified to join the components. The intermediate material may be transformable by chemical reaction, thermal conditioning, and/or other suitable mechanisms. In particular, the intermediate material may be transformable by polymerization, dissolution, drying, melting, photo curing, and/or other processes and combinations thereof. For example, the intermediate material may include a two-part acrylic cement that forms a viscous fluid initially and then polymerizes into a solid mass.

The modular components may be made of biocompatible materials including titanium, cobalt chromium steel, stainless steel, tantalum, ceramics, polymers, and/or other suitable biocompatible materials and alloys and combinations thereof.

The modular components may include an indexing mechanism for aligning the components in a predetermined relative orientation prior to the intermediate material joining the components together. The indexing features may include projections and corresponding depressions. In particular, the indexing features may include pins, pegs, bosses, rails, undulations, holes, grooves, and/or other suitable features and combinations thereof. For example, one component may include one or more pegs projecting outwardly and another component may include one or more corresponding depressions for receiving the pegs to orient the components in a desired orientation while a fluid state intermediate material transitions to a solid state.

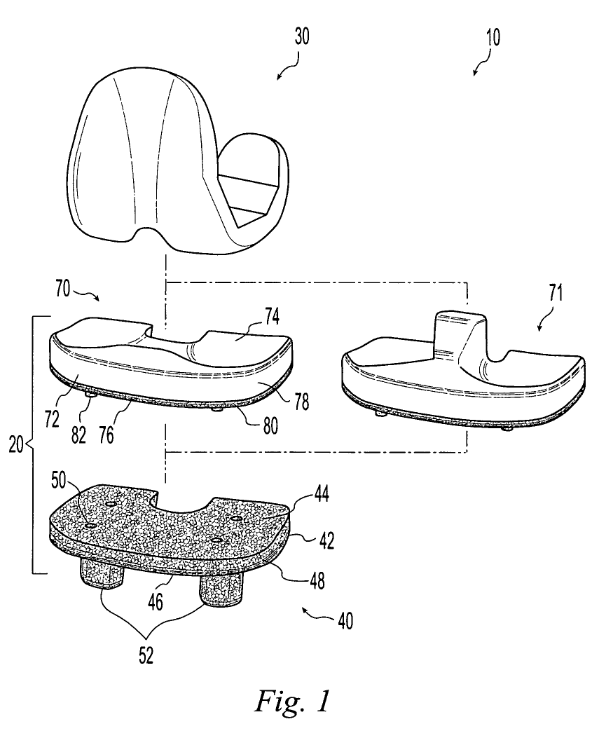

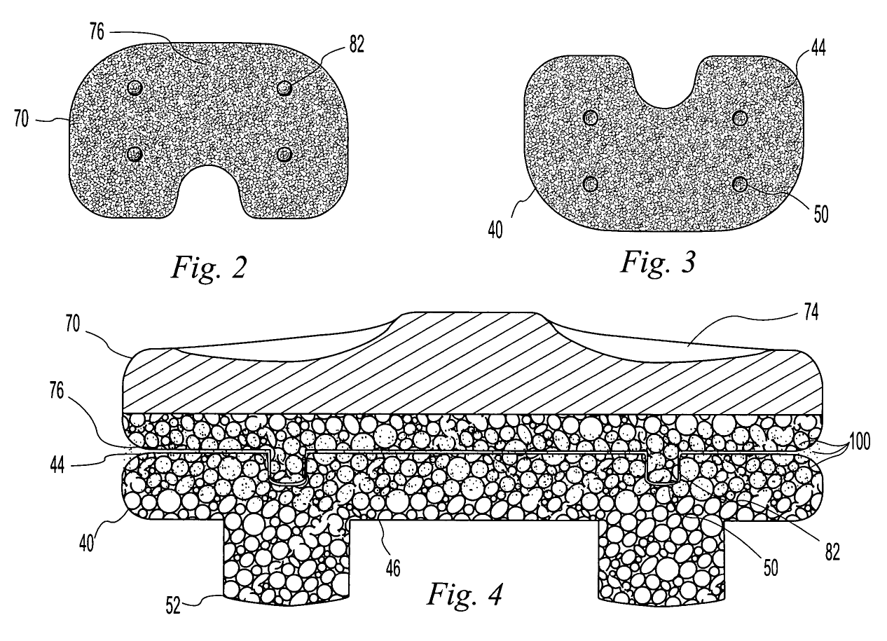

FIGS. 1-5 depict an illustrative modular porous implant in the form of a tibial implant 20 of a knee joint implant system 10. The tibial implant 20 articulates with a femoral implant 30 implanted on the end of the femur in order to restore knee joint function. The tibial implant 20 is a modular construct including a tibial tray component 40 and an articular surface component 70. The modularity of the tibial implant 20 permits the separate tray 40 and articular surface component 70 to be implanted through a smaller incision than they could be implanted through if they were a unitary construction to facilitate minimally invasive surgical techniques. In addition, the modularity of the tibial implant 20 permits intraoperative assembly of a variety of combinations of differently configured components 40, 70. In the illustrative example of FIG. 1, the articular surface component 70 is depicted as a posterior cruciate retaining implant. Other configurations of kinematic constraint including a posterior cruciate sacrificing version 71 may also be provided. The articular surface component 70 may be provided in other suitable configurations including with different heights, widths, and depths. Likewise the tibial tray component 40 may be provided in different configurations such as different heights, widths, depths, and bone engagement geometry.

The tibial tray component 40 includes a body 42 having generally planar upper and lower surfaces 44, 46 and a peripheral side wall 48 bounding the body. One or more fixation posts 52 may extend from the lower surface 46 to engage the tibial bone to aid fixation of the tray 40 to the tibia. At least the upper surface 44 includes a porous structure. Preferably both the upper and lower surfaces 44, 46 include a porous structure. In the illustrative embodiment, the body 42 is a unitary construction having pores throughout the entire body 42 similar to the porous structure of natural trabecular bone. This material is described in U.S. Pat. No. 5,282,861 entitled “OPEN CELL TANTALUM STRUCTURES FOR CANCELLOUS BONE IMPLANTS AND CELL AND TISSUE RECEPTORS”, issued to R. B. Kaplan and assigned to Ultramet. The entire disclosure of the '861 patent is incorporated herein by reference. The material is fabricated of tantalum using vapor deposition. Zimmer, Inc., with manufacturing facilities in Warsaw, Ind., sells a line of surgical implants incorporating this trabecular metal technology. The trabecular metal consists of interconnecting pores resulting in a structural biomaterial that is 80% porous and which allows much greater bone ingrowth compared to conventional porous coatings and much greater shear strength. In addition, the trabecular metal possesses a high strength-to-weight ratio. The compressive strength and elastic modulus of this trabecular metal are more similar to bone than are other prosthetic load-bearing materials. The material's low stiffness facilitates the transfer of joint loads from the upper surface 44 through the body 42 to the lower surface 46 while maintaining a physiologic load distribution across the lower surface 46 to minimize stress shielding and promote bone ingrowth into the lower surface 46.

The articular surface component 70 includes a body 72 having upper and lower surfaces 74, 76 and a peripheral side wall 78. The upper surface 74 defines a bearing surface for supporting articulation with the femoral implant 30. The upper surface 74 may be made of any suitable bearing material, including polymers, metals, and/or ceramics. Preferably the material is sufficiently elastic to permit the transfer of joint loads from the upper surface 74 through the body 72 to the lower surface 76 while maintaining a physiologic load distribution across the lower surface 76. In the illustrative embodiment, the upper surface is made of ultrahigh molecular weight polyethylene (UHMWPE). The lower surface 76 includes a porous structure. The porous structure may be formed directly in the body 72 of the articular surface component 70 or it may be applied as a separate component. In the illustrative embodiment, the lower surface 76 includes a generally planar porous layer 80 made from the same trabecular metal as the tibial tray component 40. The modulus of the trabecular metal porous layer 80 facilitates the transfer of physiologic joint loads. The porous layer 80 may be joined to the body 72 in a variety of ways including molding, melting, and/or other suitable processes. In the illustrative example, the porous layer 80 is joined to the body 72 by molding the body 72 to the porous layer 80 such that the body 72 interdigitates with the porous layer 80 by penetrating part-way through the porous layer 80. The pores at the lower surface 76 are left open. A process for compression molding such a construct is disclosed in U.S. Pat. No. 6,087,553 issued to Cohen et al. and assigned to Implex Corporation. The entire disclosure of the '553 patent is incorporated herein by reference.

The articular surface component 70 is joined to the tibial tray component 40 by interposing an intermediate material between the components so that it simultaneously interdigitates with the porous upper surface 44 of the tibial tray component 40 and the porous lower surface 76 of the articular surface component 70 as shown in FIG. 4. In the illustrative example, the intermediate material is an acrylic bone cement 100 such as is commonly used in orthopaedic surgical procedures to anchor implants in bone cavities. The cement 100 is depicted schematically in FIG. 4 by a stippling pattern within the porous surfaces 44, 76. The cement 100 includes a liquid monomer component and a powdered polymer component. When the two components are mixed, they initially form a viscous liquid that will penetrate the pores of the porous surfaces 44, 76 of the modular implant. The cement 100 is sufficiently viscous in the liquid state that it does not penetrate all the way through the tibial tray component 40, thus leaving open pores within the lower surface 46 and fixation posts 52 for boney ingrowth. After a few minutes, the cement 100 polymerizes into a solid mass. The solid cement 100 is mechanically bound in the pores of the porous surfaces 44, 76 and thus joins the articular surface component 70 to the tibial tray component 40. The cement 100 has a relatively low modulus such that it facilitates the transfer of joint loads from the articular surface component 70 to the tibial tray component 40.

An optional indexing mechanism is provided to aid in aligning the articular surface component 70 on the tibial tray component 40. The indexing feature includes one or more pegs 82 projecting from the lower surface 76 of the articular surface component 70 and one or more corresponding holes 50, or depressions, formed in the upper surface 44 of the tibial tray component 40. The pegs 82 engage the holes 50 to aid in aligning the articular surface component 70 and tibial tray component 40 in a predetermined orientation and maintaining them in that orientation until the cement 100 hardens. While a peg 82 and hole 50 indexing mechanism has been depicted having a peg 82 on the articular surface component 70 and a hole 50 on the tibial tray component 40, the gender may be reversed between the two parts and other indexing mechanisms may be used.

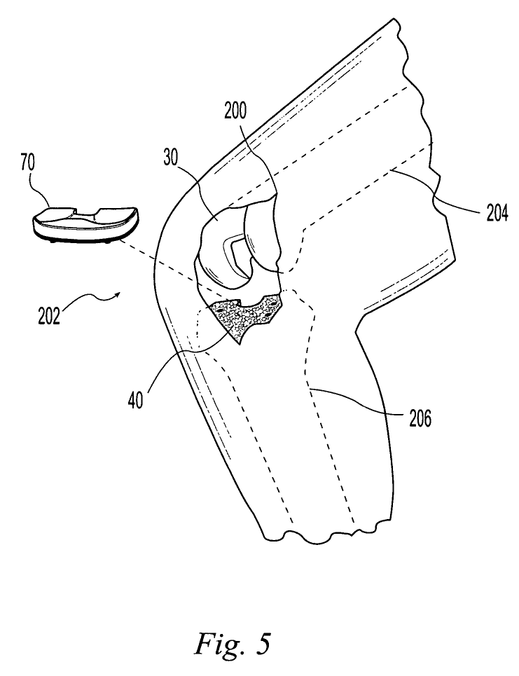

The modular porous implant of the present invention may be used in a minimally invasive surgical technique. By providing the tibial implant 20 as two separate modular components able to be assembled in the patient's body, the incision can be smaller than would be required if it were supplied as a unitary implant or a modular implant assembled outside of the patient's body. As shown in FIG. 5, an incision 200 is formed to access the knee joint 202. The femur 204 and tibia 206 are prepared to receive the implants by cutting away the ends of the bones adjacent the knee joint 202. The tibial tray component 40 is inserted through the incision 200 and positioned on the tibia 206. The femoral implant 30 is inserted through the incision 200 and positioned on the femur 204. Bone cement 100 is mixed and applied to the lower surface 76 of the articular surface component 70 and the upper surface 44 of the tibial tray component 40. The articular surface component 70 is then inserted through the incision 200 and positioned between the femoral implant 30 and the tibial tray component 40. The articular surface component 70 is aligned with the tibial tray component 40 by inserting the pegs 82 into the holes 50. This may be accomplished by feel to facilitate use in a minimally invasive surgical procedure where visibility may be limited. For example, the articular surface component 70 may be initially positioned over the tibial tray component 40 and then moved on the upper surface 44 of the tibial tray component 40 until the pegs 82 are felt to engage the holes 50. The articular surface component 70 is then pressed into firm contact with the tibial tray component 40. The bone cement 100 on the two components will flow together to form a continuous mass interdigitating with the pores of both components. Upon polymerization, the cement 100 securely bonds the two components together. The relatively low modulus of the UHMWPE upper surface 74 of the articular surface component 70, trabecular metal lower surface 76, bone cement 100 intermediate material, and trabecular metal tibial tray component 40 permit joint loads to be transmitted from the femoral implant 30 to the upper surface 74 of the articular surface component 70, through the body 72, across the cement 100 interface, through the body 42 and to the tibial bone in a physiologic load distribution to minimize stress shielding and encourage bone ingrowth into the tibial tray component 40.

Although examples of a modular porous implant and its use have been described and illustrated in detail, it is to be understood that the same is intended by way of illustration and example only and is not to be taken by way of limitation. The invention has been illustrated in the form of a modular knee implant to restore joint mobility. However, the modular porous implant may be configured for use at other locations within a patient's body to perform other functions. Accordingly, variations in and modifications to the modular porous implant and its use will be apparent to those of ordinary skill in the art, and the following claims are intended to cover all such modifications and equivalents.

What is claimed is:

1. A modular porous implant comprising:
a first implant component having a first porous surface;
a second implant component having a second porous surface; and
an intermediate material intraoperatively positionable between the first and second porous surfaces in a fluid state to interdigitate with the first and second porous surfaces, the intermediate material being transitionable intraoperatively from a fluid state to a solid state to securely join the first and second implant components.

2. The modular porous implant of claim 1 wherein the first implant component further comprises a bearing surface for supporting joint articulation opposite the first porous surface.

3. The modular porous implant of claim 2 wherein the bearing surface comprises a polymer and the first porous surface comprises a porous metal.

4. The modular porous implant of claim 3 wherein the bearing surface is joined to the first porous surface by the polymer of the bearing surface interdigitating within the pores of the first porous surface.

5. The modular porous implant of claim 3 wherein the bearing surface comprises polyethylene.

6. The modular porous implant of claim 3 wherein the first porous surface comprises tantalum.

7. The modular porous implant of claim 6 wherein the first porous surface comprises tantalum having a pore structure similar to that of natural trabecular bone

8. The modular porous implant of claim 1 wherein the second implant component comprises a porous metal.

9. The modular porous implant of claim 8 wherein the second implant component comprises a unitary construction having pores throughout.

10. The modular porous implant of claim 8 wherein the second implant component comprises tantalum.

11. The modular porous implant of claim 10 wherein the second implant component comprises tantalum having a pore structure similar to that of natural trabecular bone.

12. The modular porous implant of claim 1 wherein the first and second implant components comprise an indexing mechanism for aligning the components in a predetermined relative orientation.

13. The modular porous implant of claim 12 wherein one of the first and second porous surfaces includes at least one male indexing feature extending outwardly and the other of the first and second porous surfaces includes at least one female indexing feature engageable with the male indexing features.

14. The modular porous implant of claim 13 wherein the first porous surface includes a plurality of pegs extending outwardly and the second porous surface includes a plurality of holes engageable with the pegs.

15. The modular porous implant of claim 1 wherein the intermediate material comprises an acrylic bone cement positionable between the porous surfaces in a fluid state and polymerizable into a solid mass to join the implant components.

16. The modular porous implant of claim 1 wherein the second implant component includes a third porous surface opposite the second porous surface, the third porous surface being engageable with a bone.

17. A knee joint implant system for promoting articulation of a knee joint comprising a femur and a tibia, the implant comprising:
a femoral implant; and
a tibial implant, the tibial implant comprising a tibial tray component having a lower surface engageable with the tibia to anchor the tibial implant on the tibia and a porous upper surface, a separate articular surface component having an upper surface engageable with the femoral implant in joint articulating relationship and a porous lower surface, and an intermediate material intraoperatively positionable between the tibial tray component and articular surface component in a fluid state to interdigitate with the porous upper surface of the tibial tray component and the porous lower surface of the articular surface component, the intermediate material being transitionable intraoperatively from a fluid state to a solid state to securely join the tibial tray component and articular surface component.

18. The knee joint implant of claim 17 wherein the upper surface of the tibial tray component comprises a porous metal structure, the upper surface of the articular surface component comprises a polymer bearing material, and the lower surface of the articular surface component comprises a porous metal structure in a layer joined to the upper surface of the articular surface component.

19. The knee joint implant of claim 18 wherein the porous metal structure of the tibial tray component and articular surface component comprise tantalum having a porous structure similar to the porous structure of natural trabecular bone such that joint loads are transmitted from the femur, through the femoral implant, through the upper surface of the articular surface component, through the porous metal structure of the articular surface component, through the intermediate material, through the upper surface of the tibial tray component, and to the tibia.

20. The knee joint implant of claim 17 wherein the tibial tray component and articular surface component comprise an indexing mechanism for aligning the components in a predetermined relative orientation, the indexing mechanism including a plurality of pegs extending downwardly from the lower surface of the articular surface component and a plurality of holes formed in the upper surface of the tibial tray component for receiving the pegs.

21. A method for assembling a modular porous implant, the method comprising the steps of:
intraoperatively positioning an intermediate material between first and second implant components;
interdigitating the intermediate material in a fluid state with a porous surface on each of the first and second implant components; and
intraoperatively transitioning the intermediate material from a fluid state to a solid state to join the first and second implant components.

22. The method of claim 21 further comprising before the step of intraoperatively positioning an intermediate material between the first and second implant components, the steps of:
engaging a femoral knee implant with a prepared femur;
engaging the first implant in the form of a tibial tray component with a prepared tibia; and
providing the second implant in the form of an articular surface component.

23. The method of claim 22 wherein the tibial tray component includes a porous metal structure and the articular surface component includes a porous metal structure opposite a polymer bearing surface.
